(19) United States
(12) Patent Application Publication
(10) Pub. No.: US 2004/0055603 A1
(43) Pub. Date: Mar. 25, 2004

(54) Intra-nasal filter device

(76) Inventor: Carol Jean Emery Bruce, San Anselmo, CA (US)

Correspondence Address: Alfred F. Hoyte, Esq., 7734 16th Street N.W., Washington, DC, 20012, US

(21) Appl. No.: 10/251,868

(22) Filed: Sep. 23, 2002

(51) Int. Cl. A61M 16/00; A62B 18/02

(52) U.S. Cl. 128/206.11; 128/206.18

(57) ABSTRACT

A nasal filter device which is sized for containment within a single nostril of a human or mammal. The device has a tapered main body with a correspondingly tapered inner passage. The main body is preferably formed from a compressible material such as silicone, the material being sufficiently malleable to conform to the inner surfaces of the nostril. The inlet end of the main body is normally substantially annular but sufficiently compressible for sealing engagement with the edges of the nasal orifice. An extraction handle is provided at the outlet end to facilitate extraction of the device. The extraction handle may be hinged and sized for snap fit engagement with an annular recess provided at the inlet end of the device. A key feature of the invention is the use of a series of progressively larger annular ribs to positionally stabilize the device. The ribs are vertically spaced along the length of the device, with the first and largest rib extending radially about the inlet end. At least two types of removable filters are adapted for insertion into the interior portion of the device.

### BACKGROUND OF THE INVENTION

1. Field of the Invention

The present invention concerns filtration devices. More particularly, the invention is directed to a nasal filter device for filtering particulate matter such as smoke, dust, bacteria, and pollen which is suspended in the air.

2. Description of the Prior Art

Many humans and animals are affected adversely by airborne particulate matter and pollutants suspended in the air. Dust, pollen, viruses, bacteria, smoke particles, and other allergens are nearly always present in the airstream whether indoors or outdoors, and in order to reduce the effects thereof, many products and devices have been employed. Filtration systems are often used in combination with HVAC systems to filter the pollutants that enter interior environmentally controlled spaces, but these filtration systems cannot be used in interior spaces which do not have ventilation systems, and, of course, cannot be used outdoors. Thus an individual must resort either to drugs or other prophylactic means, or use personal filtration devices. The simplest personal filtration device are the commonly used ribbed particle mask. While somewhat effective these devices suffer from major drawbacks. First, they are highly conspicuous so that users are uncomfortable wearing them in public. Second, as they cover the entire mouth and nasal passages, the user's speech is difficult to discern. Finally, the filters are not contoured to fit to a human face and thus allow unfiltered air to enter the nasal and oral passages.

One approach that has been taken to solve the problem of unsightly and inefficient face masks is to use nasal filters. These filters are sized for insertion entirely into a single nostril, with the user employing one filter per nostril. These devices are generally effective but suffer from two major drawbacks. First, as the device must remain snugly in place to minimize air entering the nasal passages without passing through the filter element, they are often difficult to remove. The problem can be exacerbated if the user has sensitive tissues in the nasal passages or excessive nasal hair. Second, in the event of any significant facial impact, such as an air bag deployment, the device may become irretrievably lodged in the nasal passages, requiring the assistance of a physician or trained professional.

### SUMMARY OF THE INVENTION

Briefly, the invention comprises a nasal filter device which is sized for containment within a single nostril of a human or mammal. The device has a tapered main body with a correspondingly tapered inner passage. The main body is preferably formed from a compressible material such as silicone, the material being sufficiently malleable to conform to the inner surfaces of the nostril. The inlet end of the main body is normally substantially annular but sufficiently compressible for sealing engagement with the edges of the nasal orifice. An extraction handle is provided at the outlet end to facilitate extraction of the device. The extraction handle may be hinged and sized for snap fit engagement with an annular recess provided at the inlet end of the device. A key feature of the invention is the use of a series of progressively larger annular ribs to positionally stabilize the device. The ribs are vertically spaced along the length of the device, with the first and largest rib extending radially about the inlet end. At least two types of removable filters are adapted for insertion into the interior portion of the device.

It is a major object of this invention to provide an improved intra-nasal filter device.

It is another object to provide an improved intra-nasal filter device which has multiple removable filter elements.

It is another object to provide an improved intra-nasal filter device having means to prevent accidental dislodgement.

It is another object to provide an improved intra-nasal filter device having means to allow for easy removal.

Finally, it is a general goal of the invention to provide improved elements and arrangements thereof in an apparatus for the purposes described which is dependable and fully effective in accomplishing its intended purposes.

These and other objects of the present invention will become readily apparent upon further review of the following specification and drawings.

The present invention meets or exceeds all the above objects and goals. Upon further study of the specification and appended claims, further objects and advantages of this invention will become apparent to those skilled in the art.

### BRIEF DESCRIPTION OF THE DRAWINGS

Various other objects, features, and attendant advantages of the present invention will become more fully appreciated as the same becomes better understood when considered in conjunction with the accompanying drawings, in which like reference characters designate the same or similar parts throughout the several views, and wherein:

FIG. 1 shows a side view, partly in section of the device of the invention placed in a human nose.

FIG. 2 shows a cross section of the device illustrating the relation of the filter element to the main body of the device.

FIG. 3 shows a contoured alternative embodiment for the main body of the device.

FIG. 4 shows an end view of the device illustrating an alternative configuration for the extraction ring.

FIG. 5 shows a side view, partly in section, illustrating the embodiment shown in FIG. 4.

### DETAILED DESCRIPTION OF THE PREFERRED EMBODIMENTS

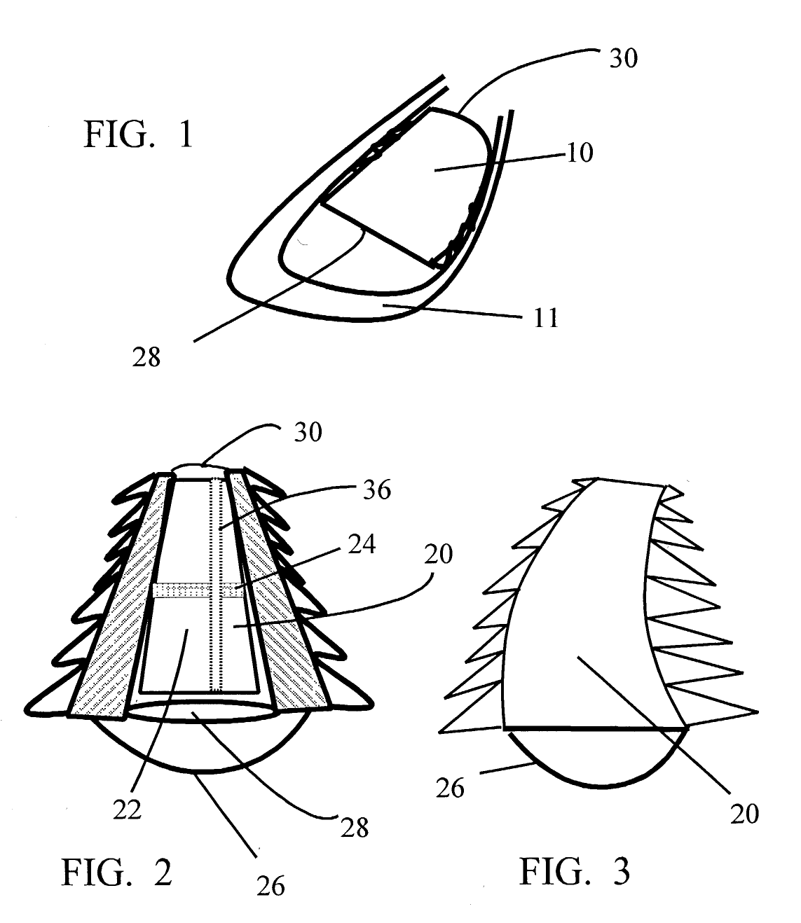

Referring now to FIGS. 1-5 the device of the present invention, generally indicated by the numeral 10, is shown. It can be seen that the device 10 has a tapered main body 20 with a central air passage 22 to allow air from the airstream to be inhaled through the users nostril 11 only after passing through a filtration element 24. An extraction ring 26 allows for facile extraction of the device 10 once it is positioned within the nostril.

The main body 20 of the device 10 is preferably made of a silicone material. Alternatively, a plastic material may be used. The material must be sufficiently malleable and compressible to conform to the inner surfaces of the nostril. The inlet end 28 of the device 10 preferably has a somewhat thicker cross section than other portions of the main body 20, giving the material forming the main body a tapered profile of decreasing cross section from inlet end 28 to outlet end 30. This configuration allows for an even more pliable outlet end 30, decreasing the discomfort to the wearer and ensuring a better fit to the contour of the inner surfaces of the nostril.

While the main body 20 is pliable, it may initially be precontoured to further reduce any discomfort experienced by the user as shown, for example, in FIG. 3. While not limited to this particular configuration, a precontoured main body can be selected by the user in accordance with the shape of his nose. In this manner, crimping or bunching of the material of the main body 20 is avoided. It can be appreciated that crimping of the material within the nasal passage would affect the effective diameter of the central air passage 22 possibly reducing the air passage therethrough.

A key feature of the invention is the provision of means to prevent unintended displacement or dislodgement of the device 10. Prior devices suffered from the problem in that any type of facial impact had the potential to dislodge the device, as could sneezing. The device could either be forced out of the nostril, creating an unsightly and potentially embarrassing situation. Even worse, in the event of a facial impact scenario, is the possibility that the device could get lodged much further up the nasal passage than intended, injuring the user and making extraction difficult. In order to reduce the incidence of accidental dislodgement or displacement of the device, and to enhance sealing engagement of the device within the user's nostril, the present invention employs a series of concentric, axially spaced, annular ribs 34. The ribs 34, which increase in diameter from the inlet end 28 to the outlet end 30, are radially extended from the main body 20. Preferably, the ribs 34 and main body 20 are molded simultaneously from a single quantity of silicone so that the device 10 is of unitary construction. Of course, a single rib 34 could be employed, but multiple ribs 34 are preferably used.

In addition to the ribs 34, at least one relatively rigid post 36, embedded in the material of main body 20, may optionally be included. The post 36 may be formed of the same material as the main body 20, but a denser and more rigid configuration is required. In the event of facial impact, the post's relatively rigid and straight shape will tend to keep the device 10 in position, effectively lodging the device 10 in place.

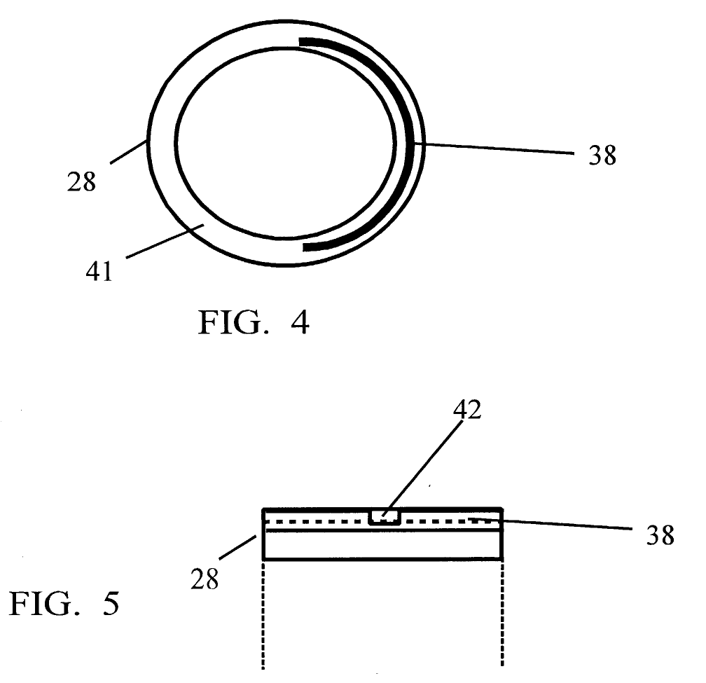

Extraction of the device is facilitated by an extraction handle or pull ring 38. The pull ring 38 may be a semicircular rigid rod which depends from opposing sides of the inlet end 28 of the device. The pull ring 38 may be either hingedly or rigidly attached to the inlet end. An alternative configuration, shown in FIG. 4, employs a snap fit arrangement for the ring 38 which is normally hidden from view, wedged into annular groove 41 formed in inlet end 28 by friction fit. This configuration eliminates the possibility of the ring 38 protruding from the user's nose. A small indentation 42 may be provided about the lip 44, which lip is formed of one side of an annular groove 41 in inlet end 28 within which ring 38 is seated. The indentation 42 is sized to allow a fingernail to pry the ring 38 from its frictional engagement within the groove 41.

At least two filter elements 24 are provided. A fine filter element 24 for fine particle entrapment such as viruses, bacteria, etc., and a large particle filter element 24 for pollen, dust, mold spores, and other large particles. The fine filter element may be a Surgikos® filter. Regardless of the type of filter 24 employed, the filter 24 is essentially disc shaped and of limited thickness relative to the length of the central air passage 22. A problem with prior art devices is that the filter thickness limits flow through the user's nasal passages. This problem is avoided through the use of essentially disc shaped filter elements 24, with the air flow perpendicular to the flat surfaces of the disc.

In use, the user first selects the desired filter element 24 and inserts it into passage 22. Of course, the device 10 may come with a filter element 24 pre-loaded. The user then places the device 10 in her nostril, ensuring a snug fit before repeating the process for the next nostril. It should be noted that in addition to the variety of contours as described above, the device 10 preferably is provided in a wide range of sizes to accommodate persons having a wide range of nostril sizes.

From the foregoing description, one skilled in the art can easily ascertain the essential characteristics of this invention and, without departing from the spirit and scope thereof, can make various changes and modifications of the invention to adapt it to various usages and conditions.

It is to be understood that the present invention is not limited to the sole embodiment described above, but encompasses any and all embodiments within the scope of the following claims:

I claim:

1. An intra-nasal filtration device for placement in the nostril of a human or mammal comprising:
a longitudinally extending main body having an axially extending central passage formed therein, the main body having an inlet end and an outlet end, said inlet end compressible to conform to interior contours of said nostril, as is said outlet end, the material of said main body having a tapered thickness, reducing in the direction of said outlet end;
at least one annular rib extending from said main body; and,
a filtration element removably inserted in said passage, adapted for frictional engagement with inner surfaces of said passage.

2. The device of claim 1 including a relatively non-compressible post embedded in, and extending in the longitudinal direction of said main body.

3. The device of claim 1 wherein said filtration element is substantially disc shaped.

4. The device of claim 3 wherein said filtration element is axially aligned with said central passage.

5. The device of claim 1 wherein a pull ring is attached to said outlet end.

6. An intra-nasal filtration device for placement in the nostril of a human or mammal comprising:
a longitudinally extending main body having an axially extending central passage formed therein, the main body having an inlet end and an outlet end, said inlet end compressible to conform to interior contours of said nostril, as is said outlet end, the material of said main body having a tapered thickness, reducing in the direction of said outlet end;
a series of annular ribs extending from said main body; and,
a filtration element removably inserted in said passage, adapted for frictional engagement with inner surfaces of said passage.

7. The device of claim 6 including a relatively non-compressible post embedded in, and extending in the longitudinal direction of said main body.

8. The device of claim 6 wherein said filtration element is substantially disc shaped.

9. The device of claim 8 wherein said filtration element is axially aligned with said central passage.

10. The device of claim 6 wherein a pull ring is attached to said outlet end.
